(19) United States
(12) Patent Application Publication
(10) Pub. No.: US 2022/0104998 A1
(43) Pub. Date: Apr. 7, 2022

(54) SURGICAL TABLE AND METHOD FOR DETECTING CARDIOPULMONARY RESUSCITATION BY THE SURGICAL TABLE

(71) Applicant: TRUMPF MEDIZIN SYSTEME GMBH + CO. KG, Saalfeld (DE)

(72) Inventor: Matthias JÄGER, Rudolstadt (DE)

(21) Appl. No.: 17/494,882

(22) Filed: Oct. 6, 2021

(30) Foreign Application Priority Data
Oct. 7, 2020 (EP) 20200574.0

(51) Int. Cl. A61H 31/00; A61G 13/10; G16H 40/63; G16H 10/60

(52) U.S. Cl. CPC A61H 31/008; A61G 13/10; A61H 2201/5061; G16H 10/60; G16H 40/63

(57) ABSTRACT

A surgical table comprises a base, a tabletop attached to the base, a force sensor, and a controller. The force sensor is configured to detect a force exerted to the tabletop (3) and to transmit the force to the controller. The controller is configured to evaluate the force exerted to the tabletop and to execute a predefined routine for determining cardiopulmonary resuscitation for a patient lying on the tabletop by distinguishing forces caused by the cardiopulmonary resuscitation from other forces exerted to the tabletop.

### CROSS-REFERENCE TO RELATED APPLICATIONS

This application claims the benefit of European Patent Application Serial No. EP20200574.0, filed Oct. 7, 2020, the entire disclosure of which is incorporated herein by reference in its entirety.

### BACKGROUND

The present disclosure relates to a surgical table and a method for detecting cardiopulmonary resuscitation by the surgical table, in particular, to a surgical table and a method for detecting cardiopulmonary resuscitation by the surgical table in an automatic manner. Nowadays, there is a corporate strategy of connected care. This means that relevant data of a patient are collected, e.g., during hospitalization and provided to medical staff if necessary. However, collecting of some of these patient data is cumbersome and, therefore, it is not logged immediately so that there is the risk that it is omitted. Therefore, there is a need to provide an apparatus and a method for automatically collecting patient's data, in particular, occurrence of a cardiopulmonary resuscitation (CPR) during a surgical intervention. The need is achieved by a surgical table and method disclosed herein.

### SUMMARY

The present disclosure includes one or more of the features recited in the appended claims and/or the following features which, alone or in any combination, may comprise patentable subject matter.

According to an aspect of the present disclosure, a surgical table comprises a base, a tabletop attached to the base, a force sensor, and a controller. The force sensor is configured to detect a force exerted to the tabletop and to transmit the force to the controller, and the controller is configured to evaluate the force exerted to the tabletop and to execute a predefined routine for determining cardiopulmonary resuscitation for a patient lying on the tabletop by distinguishing forces caused by the cardiopulmonary resuscitation from other forces exerted to the tabletop.

By the surgical table having these features, information on when cardiopulmonary resuscitation is performed on a patient can be gathered as patient's data in an automatic manner without any further manual intervention and in a reliable manner since other forces not caused by the cardiopulmonary resuscitation are not considered by the routine for determining cardiopulmonary resuscitation.

The force sensor is preferably configured to output a signal indicative of the detected force, the force signal being transmitted to the controller.

In some embodiments, the predefined routine is configured to stop evaluation during motion of the surgical table. In such an embodiment, the controller may be configured to receive one or more inputs indicative of motion of the surgical table. The inputs may be one or more of: signals from, or relating to, control drives of the surgical table, the control drives being configured to move the tabletop; and signals from wheel sensors configured to detect motion of wheels supporting the surgical table. Due to this feature, detection of a pattern of forces caused by the motion of the surgical table which might be similar to a pattern of forces caused by cardiopulmonary resuscitation can be suppressed so that it is not considered as being the cardiopulmonary resuscitation.

In some embodiments, the predefined routine is configured to determine exerted forces as forces of the cardiopulmonary resuscitation when force peaks values are within a predefined force peak value range and a force peak interval is within a predefined force peak interval range. Since the force peaks values caused by cardiopulmonary resuscitation are usually similar and a frequency of the force peaks is almost constant, due to the definition of the force peak value range and the force peak interval range, cardiopulmonary resuscitation can be determined reliably.

In some embodiments, the predetermined force peak value range is defined by an upper limit value being 5% more than a first detected force peak value and a lower limit value being 5% less of the first detected force peak value. The definition of the force peak value range in such a manner enhances the reliability of the determination of the cardiopulmonary resuscitation.

In some embodiments, the predetermined force peak interval is formed by a range of 80 to 120 force peaks/minute. The definition of the force peak interval range in such a manner enhances the reliability of the determination of the cardiopulmonary resuscitation.

In some embodiments, the force sensor may be arranged in the tabletop. When the force sensor is arranged in the tabletop, a direct detection of the exerted force without any influence of the inertia of the tabletop is possible in order to enhance the detection of the force exerted by the cardiopulmonary resuscitation.

In some embodiments, the force sensor may be arranged in the base of the surgical table. When being arranged in the base of the surgical table where usually the controller of the surgical table is located, connecting of the force sensor and the controller can easily be performed and it can be considered as highly reliable.

In some embodiments, the force sensor may be configured as a sensor additionally providing load data of a quasi-static load of the tabletop to the controller. When the force sensor being provided for additionally supplying quasi-static load data to the controller is used, no additional force sensor for the determination of the cardiopulmonary resuscitation is necessary in order to reduce effort and costs.

In some embodiments, the controller may be configured to store occurrence of the cardiopulmonary resuscitation. When the controller is configured to store the occurrence of the cardiopulmonary resuscitation, the medical staff is exempt from the need of manually logging the cardiopulmonary resuscitation.

In some embodiments, the controller may comprise at least one processor, and memory encoding instructions which, when executed by the at least one processor, cause the at least one processor to be configured to carry out the steps described herein. Where the controller is configured to store data, the controller further comprises storage memory, preferably non-volatile memory.

According to a further aspect of the present disclosure, a method for detecting cardiopulmonary resuscitation by a surgical table includes the steps: detecting several consecutive force peaks by the force sensor and determining the consecutive force peaks as cardiopulmonary resuscitation if the force peak values are within a predefined force peak value range and the force peaks are within a predefined force peak interval range. By this method, cardiopulmonary resuscitation may be gathered as patient's data in an automatic manner without any further manual intervention.

In some embodiments, the predetermined force peak value range may be defined by an upper limit value being 5% more than a first detected force peak value and a lower limit value being 5% less than the first detected force peak value. The execution of the method with the force peak value range in such manner enhances the reliability of the determination of the cardiopulmonary resuscitation.

In some embodiments, an end of the cardiopulmonary resuscitation is determined if, within a predefined interval after a last force peak determined as being a force peak of the cardiopulmonary resuscitation, there are no force peaks, or the consecutive force peaks are outside the predefined force peak value range and/or outside the predefined force peak interval range. By these features of the method, cardiopulmonary resuscitation, in particular, a duration of the cardiopulmonary resuscitation may be gathered as patient's data in an automatic manner without any further manual intervention since also the end of the cardiopulmonary resuscitation is detected.

In some embodiments, occurrence of the cardiopulmonary resuscitation may be stored by the controller. When storing the occurrence of the cardiopulmonary resuscitation by the controller, the medical staff is exempt from the need of manually logging the cardiopulmonary resuscitation.

In some embodiments, a duration of the cardiopulmonary resuscitation may be detected and stored by the controller. When the duration of the cardiopulmonary resuscitation is detected and stored, severity of an incident of a cardiac arrest may be estimated retrospectively.

In some embodiments, the occurrence of the cardiopulmonary resuscitation and/or a duration of the cardiopulmonary resuscitation may be transferred to a hospital's Electronic Medical Record system. When this data is transferred to the Electronic Medical Record system, the data may be available for any necessary use by the hospital's staff.

In some embodiments, the step of determining the consecutive force peaks (P1, P2, P3) may be stopped during motion of the surgical table. Due to this method feature, detection of a pattern of forces caused by the motion of the surgical table which might be similar to a pattern of forces caused by cardiopulmonary resuscitation may be suppressed so that it is not considered as being the cardiopulmonary resuscitation.

According to a yet further aspect of the present disclosure, there is provided a method for detecting cardiopulmonary resuscitation, carried out by a patient support apparatus comprising a force sensor, including the steps: detecting several consecutive force peaks (P1, P2, P3, P1′, P2′, P3′) by the force sensor; and determining the consecutive force peaks (P1, P2, P3) as cardiopulmonary resuscitation if the force peak values are within a predefined force peak value range (RFP) and the force peaks are within a predefined force peak interval range.

Any feature in one aspect of the disclosure may be applied to other aspects of the disclosure, in any appropriate combination. In particular, method aspects may be applied to apparatus aspects, and vice versa. Furthermore, features implemented in hardware may be implemented in software, and vice versa. Any reference to software and hardware features herein should be construed accordingly.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 shows a principal view of a first embodiment of a surgical table according to the present disclosure;

FIG. 2 shows a principal view of a second embodiment of the surgical table according to the present disclosure;

FIG. 3 shows a diagram including an example of a force curve of a force caused by cardiopulmonary resuscitation; and

FIG. 4 shows a diagram including an example of a force curve of a force not caused by the cardiopulmonary resuscitation.

### DETAILED DESCRIPTION

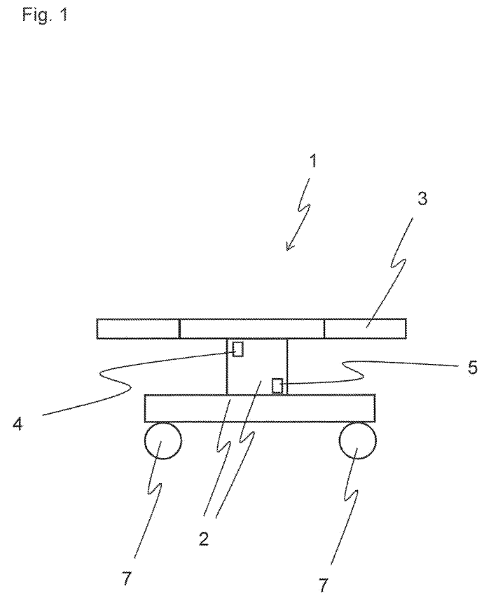

FIG. 1 shows a principal view of a first embodiment of a surgical table 1 according to the present disclosure. The surgical table 1 comprises a base 2 and a tabletop 3 attached to the base 2. The surgical table 1 is configured as a movable surgical table having castors 7 for being movable.

The surgical table 1 further comprises a force sensor 4 and a controller 5. The force sensor 4 is arranged in the base of the surgical table 1. The controller 5 comprises at least one processor, and memory encoding instructions which, when executed by the at least one processor, cause the at least one processor to be configured to carry out the steps described herein.

The force sensor 4 is further configured to detect a force exerted to the tabletop 3. This detected force can be a force caused by a patient lying on the tabletop 3, by external forces impacting onto the tabletop 3, or by inertia force when the surgical table 1 is, e.g., moved across a door sill. Moreover, the force sensor 4 is configured as a sensor additionally providing quasi-static load data of the tabletop 3 to the controller 5. This means that the force sensor 4 can be used for two different tasks, namely for the detection of the force caused by the cardiopulmonary resuscitation and for the detection of, e.g., the weight of the patient for detecting overload of the surgical table 1. In an alternative embodiment, the first sensor 4 is provided only for detecting the forces due to the cardiopulmonary resuscitation.

The controller 5 is configured to control drives (not shown) of the surgical table 1 to move the tabletop 3 with respect to the base 2 in order to perform a height adjustment of the tabletop 3 in order to tilt the tabletop 3. Further, the controller 5 is configured to evaluate the force exerted to the tabletop 3.

The force sensor 4 is configured to output a force signal, in dependence on the force exerted to the tabletop 3, and transmit that force signal to the controller.

Furthermore, the controller 5 is configured to execute a predefined routine for determining cardiopulmonary resuscitation for a patient lying on the tabletop 3. This predefined routine is configured to distinguish forces caused by the cardiopulmonary resuscitation from other forces exerted to the tabletop 3.

Moreover, the controller 5 is configured to store occurrence and duration of the cardiopulmonary resuscitation. In alternative embodiments, the controller 5 does not store the occurrence and duration of the cardiopulmonary resuscitation but transfers the occurrence and duration to, e.g., a hospital's Electronic Medical Record system (EMS). Where the controller is configured to store data relating to the occurrence and duration of the cardiopulmonary resuscitation, the controller comprises storage memory, such as non-volatile memory. In the alternative embodiments, the controller may only comprise volatile memory configured to temporarily store the data relating to the occurrence and duration of the cardiopulmonary resuscitation before transferring the data to an external device, or permanent storage facility such as a hospital's (EMS).

The predefined routine is configured to stop evaluation during motion of the surgical table 1. In an alternative embodiment, the routine does not stop evaluation during motion of the surgical table 1 but, e.g., it performs the evaluation but it does not store or transfer the result of the evaluation.

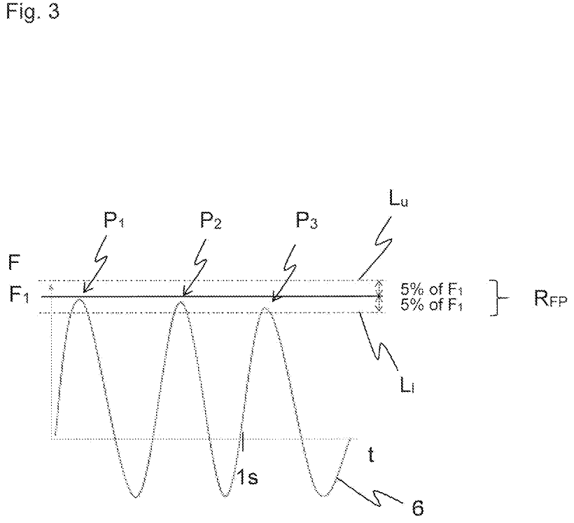

Furthermore, the predefined routine is configured to determine the exerted forces as forces of the cardiopulmonary resuscitation when force peaks values are within a predefined force peak value range RFP (see FIG. 3) and a force peak interval is within a predefined force peak interval range.

In alternative embodiments, the routine is configured not to be focused on the force peaks but, e.g., on other portions of the force curve.

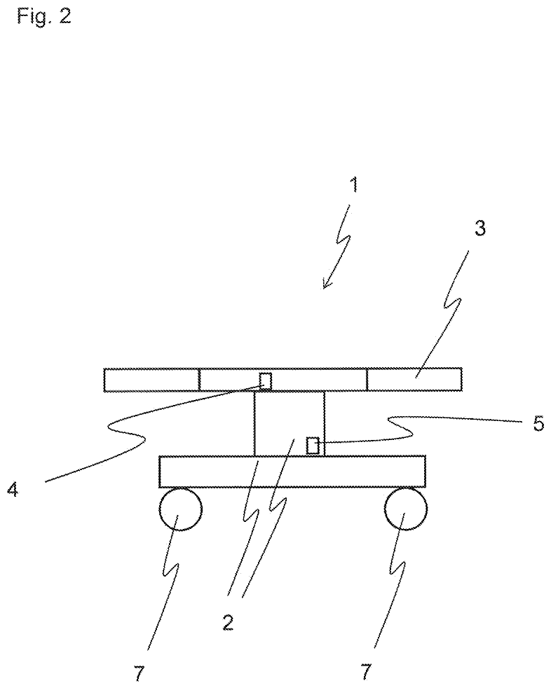

FIG. 2 shows a principal view of a second embodiment of the surgical table 1 according to the present disclosure.

The surgical table 1 according to the second embodiment differs from the surgical table 1 according to the first embodiment in that the force sensor 4 is not arranged in the base 2 but it is arranged in the tabletop 3.

FIG. 3 shows a diagram including an example of a force curve 6 of a force caused by the cardiopulmonary resuscitation. On the abscissa, an elapsed time t is plotted and, on the ordinate, a force value F is plotted.

The predetermined force peak value range RFP is defined by an upper limit value Lu being 5% more than a first detected force peak value F1 and a lower limit value Ll being 5% less than the first detected force peak value F1. In this embodiment, the predetermined force peak interval is formed by a range of 80 to 120 force peaks/minute. In alternative embodiments, the ranges are defined as having other limits.

As to be seen in FIG. 3, all values of the force peaks P1, P2, P3 of the force curve 6 are within the range RFP. Furthermore, as to be seen from the time course, two oscillations are detected within 1 second which corresponds to a force peak interval of 120 force peaks/minute. Thus, the exerted force is determined as force of the cardiopulmonary resuscitation.

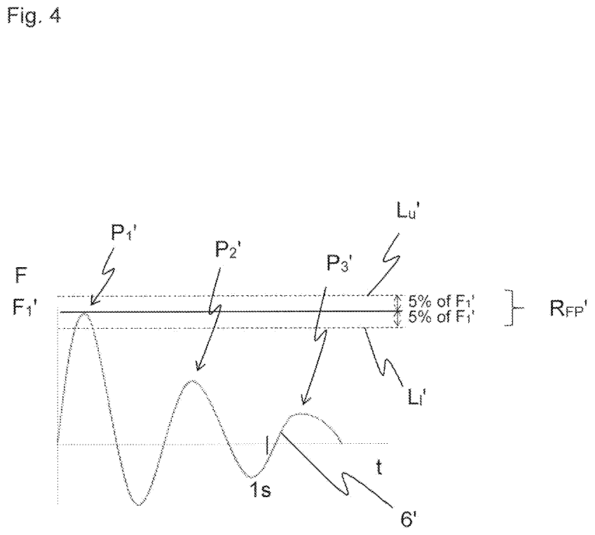

FIG. 4 shows a diagram including an example of a force curve 6′ of a force not caused by the cardiopulmonary resuscitation.

Also here, on the abscissa, the elapsed time t is plotted on and, on the ordinate, the force value F is plotted. Also, the indications concerning the upper limit value Lu′ being 5% more than the first detected force peak value F1′ and the lower limit value Ll′ being 5% less than the first detected force peak value F1′ are plotted corresponding to FIG. 3.

As to be seen in FIG. 4, the value of the consecutive force peaks P2′, P3′ following the first force peak P1′ are not within the range RFP′ having the upper limit value Lu′ and the lower limit value Ll′. Therefore, the exerted force is not determined as being a force of a cardiopulmonary resuscitation.

In use, several consecutive force peaks P1, P2, P3, P1′, P2′, P3′ are detected by the force sensor 4. The consecutive force peaks P1, P2, P3, P1′, P2′, P3′ are determined as cardiopulmonary resuscitation if their force peak values are within the predefined force peak value range RFP, RFP ‘.

The predefined force peak value range RFP, RFP’ is defined by the upper limit value being 5% more than the first detected force peak F1, F1′ and by the lower limit value being 5% less than the first detected force peak value F1, F1′. As already mentioned above, alternatively, the ranges are defined as having other limits or the routine may be configured not to be focused on the force peaks but on other portions of the force curve.

An end of the cardiopulmonary resuscitation is determined if, within a predefined interval after the last force peak determined as being a force peak of the cardiopulmonary resuscitation, there are no force peaks, or the force peak values of the consecutive force peaks are outside the predefined force peak value range RFP, RFP′ and/or outside the predefined force peak interval. Alternatively, an end of the cardiopulmonary resuscitation is not determined.

The occurrence of the cardiopulmonary resuscitation is stored by the controller 5. Alternatively, the occurrence of the cardiopulmonary resuscitation is not stored by the controller 5 but, e.g., transferred to another database.

Except the occurrence of the cardiopulmonary resuscitation, a duration of the cardiopulmonary resuscitation is detected and stored by the controller 5. Alternatively, the duration is not stored by the controller 5, but, e.g., transferred to another database.

Optionally, the occurrence of the cardiopulmonary resuscitation and/or the duration of the cardiopulmonary resuscitation are/is transferred to a hospital's Electronic Medical Record System.

While the present disclosure has been illustrated and described in detail in the drawings and the foregoing description, such illustration and description are to be considered illustrative or exemplary and not restrictive. The invention is not limited to the disclosed embodiments. From reading the present disclosure, other modifications will be apparent to a person skilled in the art. Such modifications may involve other features, which are already known in the art and may be used instead of or in addition to features already described herein. In the claims, the word “comprising” does not exclude other elements or steps, and the indefinite article “a” or “an” does not exclude a plurality.

1. A surgical table comprising
a base,
a tabletop attached to the base,
a force sensor, and
a controller, wherein
the force sensor is configured to detect a force exerted to the tabletop and to transmit the force to the controller, and
the controller is configured to evaluate the force exerted to the tabletop and to execute a predefined routine for determining cardiopulmonary resuscitation for a patient lying on the tabletop by distinguishing forces caused by the cardiopulmonary resuscitation from other forces exerted to the tabletop.

2. The surgical table of claim 1, wherein the predefined routine is configured to stop evaluation during motion of the surgical table.

3. The surgical table of claim 2, wherein the predefined routine is configured to determine exerted forces as forces of the cardiopulmonary resuscitation when force peaks values are within a predefined force peak value range (RFP) and a force peak interval is within a predefined force peak interval range.

4. The surgical table of claim 3, wherein the predetermined force peak value range (RFP) is defined by an upper limit value (Lu) being 5% more than a first detected force peak value and a lower limit value (Ll) being 5% less than the first detected force peak value (F1).

5. The surgical table of claim 3, wherein the predetermined force peak interval range is formed by a range of 80 to 120 force peaks/minute.

6. The surgical table of claim 1, wherein the force sensor is arranged in the tabletop.

7. The surgical table of claim 1, wherein the force sensor is arranged in the base.

8. The surgical table of claim 1, wherein the force sensor is configured as a sensor additionally providing load data of the tabletop to the controller.

9. The surgical table of claim 1, wherein the controller is configured to store occurrence of the cardiopulmonary resuscitation.

10. The surgical table of claim 1, wherein the force sensor is configured to output a signal indicative of the detected force, the force signal being transmitted to the controller.

11. A method for detecting cardiopulmonary resuscitation by a surgical having a tabletop, a controller, and a force sensor configured to detect a force exerted to the tabletop and to transmit the force to the controller, the controller being configured to evaluate the force exerted to the tabletop and to execute a predefined routine for determining cardiopulmonary resuscitation for a patient lying on the tabletop by distinguishing forces caused by the cardiopulmonary resuscitation from other forces exerted to the tabletop, the method including the steps:
detecting several consecutive force peaks (P1, P2, P3, P1′, P2′, P3′) by the force sensor; and
determining the consecutive force peaks (P1, P2, P3) as cardiopulmonary resuscitation if the force peak values are within a predefined force peak value range (RFP) and the force peaks are within a predefined force peak interval range.

12. The method of claim 11, wherein the predefined force peak value range (RFP) is defined by an upper limit value (Lu) being 5% more than a first detected force peak value (F1) and a lower limit value (Ll) being 5% less than the first detected force peak value (F1).

13. The method of claim 11, wherein an end of the cardiopulmonary resuscitation is determined if, within a predefined interval after a last force peak (P3) determined as being a force peak of the cardiopulmonary resuscitation, there are no force peaks, or the consecutive force peaks are outside the predefined force peak value range (RFP) and/or outside the predefined force peak interval range.

14. The method of claim 11, wherein occurrence of the cardiopulmonary resuscitation is stored by the controller.

15. The method of claim 14, wherein a duration of the cardiopulmonary resuscitation is detected and stored by the controller.

16. The method of claim 11, wherein occurrence of the cardiopulmonary resuscitation and/or a duration of the cardiopulmonary resuscitation are/is transferred to a hospital's Electronic Medical Record system.

17. The method of claim 11, wherein the step of determining the consecutive force peaks (P1, P2, P3) is stopped during motion of the surgical table.

18. A method for detecting cardiopulmonary resuscitation, carried out by a patient support apparatus comprising a force sensor, including the steps:
detecting several consecutive force peaks (P1, P2, P3, P1′, P2′, P3′) by the force sensor; and
determining the consecutive force peaks (P1, P2, P3) as cardiopulmonary resuscitation if the force peak values are within a predefined force peak value range (RFP) and the force peaks are within a predefined force peak interval range.
